(19) United States
(12) Patent Application Publication
(10) Pub. No.: US 2006/0160206 A1
(43) Pub. Date: Jul. 20, 2006

(54) Parallel prosessing of microfluidic devices

(75) Inventors: Mats Holmquist, Sollentuna (SE); Johan Engstrom, Uppsala (SE)

Correspondence Address: BIRCH STEWART KOLASCH & BIRCH, PO BOX 747, FALLS CHURCH, VA, 22040-0747, US

(73) Assignee: Gyros AB, Uppsala (SE)

(21) Appl. No.: 10/537,318

(22) PCT Filed: Dec. 1, 2003
(86) PCT No.: PCT/SE03/01850

§ 371 Date: Dec. 6, 2005

Related U.S. Application Data
(60) Provisional application No. 60/430,978, filed on Dec. 5, 2002.

(30) Foreign Application Priority Data
Dec. 2, 2002 (SE) 0203595-4

(51) Int. Cl. C12M 1/34

(52) U.S. Cl. 435/287.2

(57) ABSTRACT

Microfluidic arrangement which comprises A) a number of microfluidic devices, and B) an instrument which comprises a spinner motor and a rotary member arranged such that liquid flow can be driven centrifugal force in each of the devices by spinning the. Each of the microfluidic devices comprises microchannel structures in a common planar layer I. The characteristic feature is that layer I of each device can be oriented radially and at an angle ≠0° relative to the plane of the rotary member, with preference for 90°. The rotary member has seats for holding the devices. A microfluidic device comprising i) two essentially planar and parallel opposite sides, and edge sides, ii) a set of one, two, three or more essentially equal microchannel structures, each of which comprises a first inlet arrangement comprising an inlet port IP I₁. The characteristic feature is that a) each of the inlet ports is present in an edge side, and b) the wettability of the inner walls of said first inlet arrangement permits penetration by capillarity of at least a predetermined first volume of an aqueous liquid.

### TECHNICAL FIELD

The present invention relates to the miniaturization of analytical, synthetic, preparative etc procedures within chemical and biological sciences.

One aspect of the invention is a microfluidic instrument arrangement, which comprises a) one, two or more essentially equal microfluidic devices, and b) an instrument for processing the microfluidic devices. Additional aspects are: i) the instrument as such, ii) the use of the instrument for processing the microfluidic device (method of processing), iii) a microfluidic device as such, and (iv) a method for loading a predetermined liquid aliquot to each of the microchannel structure of a microfluidic device (“Dip-Chip technology”). The instrument may be used for processing different kinds of microfluidic devices. The microfluidic device may be processed in the innovative instrument but also by the use of other instruments and/or other means. The microfluidic device of item (iii) is adapted to the Dip-Chip technology.

A microfluidic device comprises a number of microchannel structures through which a liquid flow is used for transporting analytes, reactants etc. Devices used in the innovative arrangement/instrument utilize centrifugal force created by spinning the devices for the transport within at least a part of each microchannel structure. Devices of aspect (iii) above do not need to utilize centrifugal force for driving liquid flow.

### BACKGROUND TECHNOLOGY

Patents and patent applications cited in this specification are hereby incorporated by reference in their entirety.

Microfluidic Systems

The use of centrifugal force for moving liquids within microfluidic systems on circular platforms has been described among others by Abaxis Inc (U.S. Pat. No. 5,122,284, U.S. Pat. No. 5,591,643, U.S. Pat. No. 5,160,702, U.S. Pat. No. 5,472,603, WO 9533986, WO 9506870); Molecular Devices (U.S. Pat. No. 5,160,702); Gamera Biosciences/Tecan (WO 9721090, WO 9807019, WO 9853311), WO 01877486, WO 0187487; Gyros AB/Amersham Pharmacia Biotech (WO 9955827, WO 9958245, WO 0025921, WO 0040750, WO 0056808, WO 0062042, WO 0102737, WO 0146465, WO 0147637, WO 0147638, WO 0154810, WO 0241997, WO 0241998, WO 0275312, WO 274438, WO 0275775, WO 0275776, WO 03018198, WO 03024598 and WO 03093802 (SE0201310-0).

Centrifugal force has also been used for sector-shaped microfluidic discs. See WO 9607919 (Biometric Imaging).

WO 0173396 (Caliper) describes a microfluidic device in which there are inlet ports designed as capillary tips.

Non-Microfluidic Systems

Chromatographic columns have been placed in centrifugal rotors and spinning used for driving samples and other liquids through the columns.

Sedimentation in packed columns that are oriented parallel to each other in the same radial direction in a centrifugal field has been utilized for performing blood tests. See for instance U.S. Pat. No. 6,114,179 and U.S. Pat. No. 5,338,689 (Stiftung für diagnostische Forschung).

Other non-microfluidic systems based on circular discs that can be spun have been described in U.S. Pat. No. 4,469,973 (Guigan), U.S. Pat. No. 4,519,981 (Guigan), U.S. Pat. No. 4,390,499 (IBM), EP 392475 (Idemitsu) and many others.

Problems Associated with Prior Microfluidic Techniques Utilizing Centrifugal Force.

Procedures within chemical and biological sciences have been adapted to miniaturized formats in order to increase the productivity of performing analytical, synthetic, preparative etc. There is a general desire to increase a) the total number of successful tests per time unit, per test device and per instrument, and b) the number of successful tests/results from a given volume/amount of sample, reagent, etc.

Miniaturization often creates new problems and/or accentuates problems that are easy to handle in larger systems. Interfacing to a microfluidic device is more difficult the smaller and/or more dense-packed the microchannel structures are. The risk for inaccuracies in the transfer of liquid from the instrument to the individual microchannel structures increases dramatically when going down in the μl-range, in particular when entering into its lower part (sub-μl-range or nanolitre range, nl-range including picolitre range). The significance of losses caused by undesired evaporation and by irregular adherence to surfaces increases dramatically. Intermolecular forces become more important that may lead to a liquid behaviour that is different compared to larger scales. In total technical solutions that are applicable to the macroworld many times are not always applicable to the microworld. New technical solutions and modifications are required.

### OBJECTS OF THE INVENTION

Objects of the invention are to provide an instrument for processing of identical or different microfluidic devices, which enables:
a) parallel processing of larger numbers of microchannel structures and/or microfluidic devices; and/or
b) enhanced versatility for the user with respect to the number and the type of microfluidic devices that can be processed in parallel; and/or
c) parallel storage of similar and/or dissimilar liquid volumes in individual microchannel structures before their use in an intended procedure; and/or
d) driving a liquid flow in various directions relative to a predetermined main direction of a microchannel structure/microfluidic device, e.g. i) back and forth in a predetermined part of a microchannel structure, ii) in the main direction in one part of a structure and in some other direction, for instance opposite direction, in another part etc; and/or
e) larger numbers of process steps and process microcavities per microchannel structure; and/or
f) individual examination and/or irradiation of the devices without imperative removal of devices from the instrument; and/or
g) individual regulation of liquid flow velocity in the devices; etc.

Item d. ii) typically means that the liquid flow is in the main direction in the upstream part of a microchannel structure and in the opposite direction in a subsequent downstream part, typically the last part of the structure. The definition of “main direction” is given under the heading “Microfluidic device”.

Item f) includes that the liquid flow velocity in corresponding parts of the microchannel structures in one device may differ in a predetermined manner from the liquid flow velocity in corresponding parts of another device that is processed in parallel.

Another object is a microfluidic device that has an inlet arrangement, which simplifies a rapid, reproducible, reliable and accurate loading of well-defined minute liquid volumes to the individual microchannel structures of the device. This object in particular emphasizes volumes in the nl-range, i.e. ≦5,000 nl.

Terms, such as larger, enhanced, simplify etc, are relative to known technology.

Where appropriate these objects apply also to methods utilizing the innovative instrument, the innovative microfluidic device and/or the innovative arrangement.

These objects concern microfluidic systems in which electrokinetic and/or non-electro kinetic liquid flow is utilized. Centrifugal force or other inertia forces, or pressure differences created externally or internally within the individual microchannel structures may be used for creating non-electrokinetic liquid flow. Applying overpressure at the inlet and/or sub-pressure at the outlet of a microconduit/microchannel structure (relative to atmospheric pressure) may create useful pressure differences.

A liquid flow may be active or passive. In principle a liquid flow that is not driven solely by capillarity is active and typically created by external means. Active liquid flow includes flow driven by centrifugal force and other inertia forces, and any other force mentioned herein (except capillary force). Passive liquid flow and capillary liquid flow are in principle synonymous.

### DRAWINGS

The first digit in each reference number refers to the figure while the subsequent two digits refer to the detail concerned.

FIGS. 1a-d illustrate views of a variant of the innovative microfluidic arrangement which contains an annular arrangement of 10 microfluidic devices. FIG. 1a is a side view of the arrangement. FIG. 1b is a view from above. FIG. 1c is a slanted view from above. FIG. 1d is a view through the cross-section defined by plane A-A and illustrates that devices can be oriented radially.

FIGS. 2a-d illustrate views of a variant which contains an annular arrangement of 4 microfluidic devices. The arrangement is viewed in the same directions as in FIG. 1. FIG. 2d illustrates that the devices can be rotated, i.e. can have different orientations relative to the radius passing from the centre of the annular arrangement through the device.

FIG. 3a-d illustrates schematically a rectangular form of the innovative microfluidic device, which is constructed from several planar substrates and comprises five microchannel structures. FIGS. 3a-c are slanted views on a bottom substrate layer, an intermediate substrate layer, and a top substrate layer, respectively. FIG. 3d illustrates how these substrate layers are joined together to form the microfluidic device with its microchannel structures.

### THE INVENTION

Microfluidic Instrument Arrangement (First Aspect)

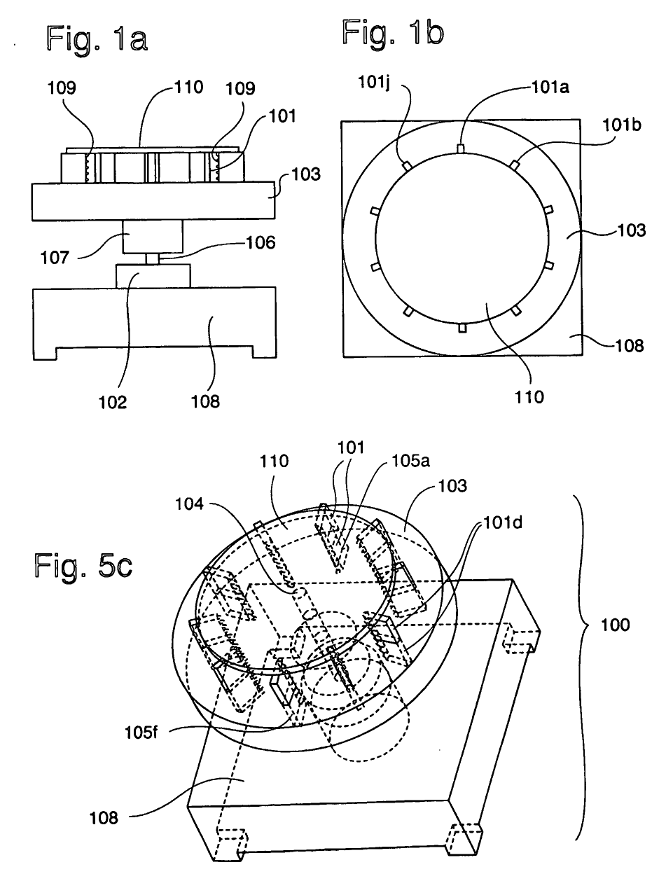

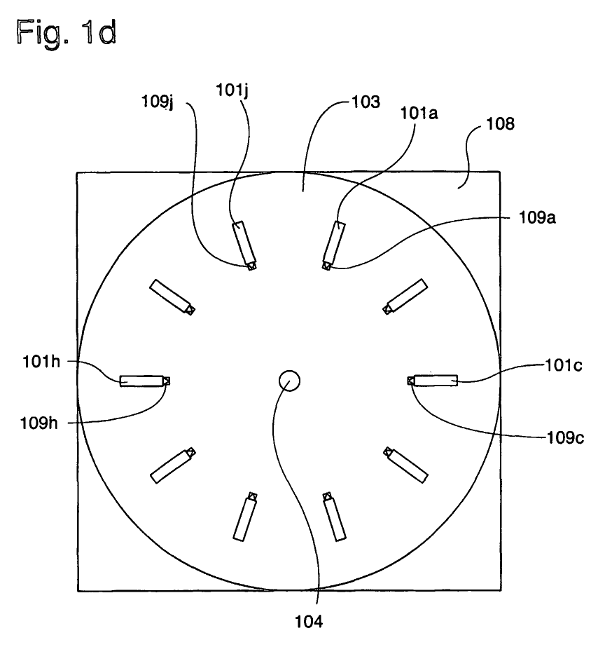

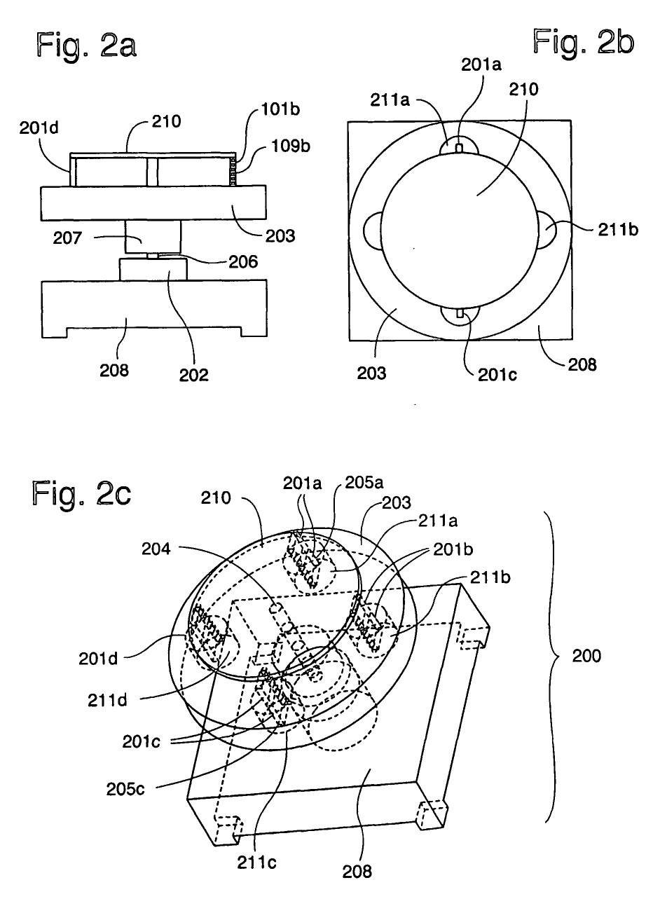

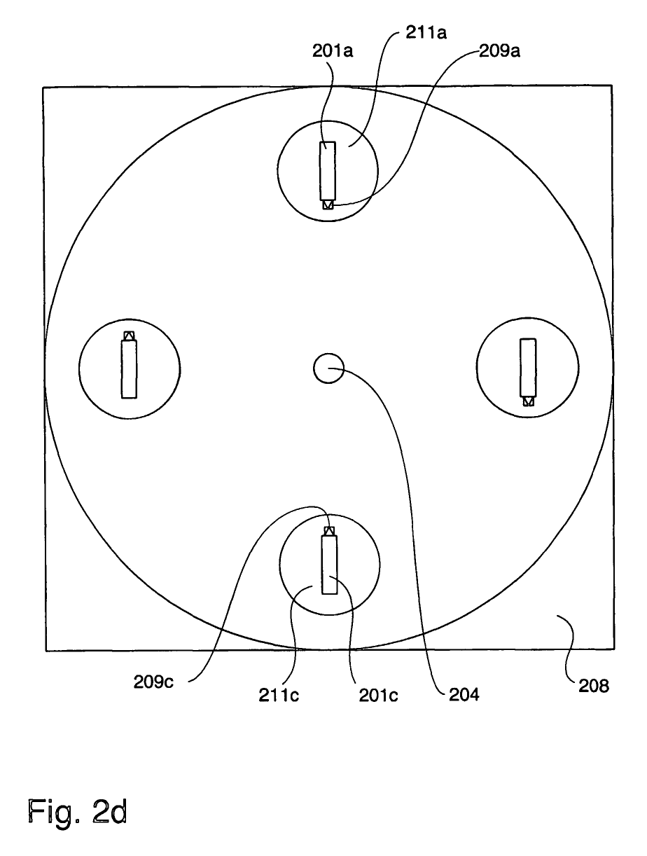

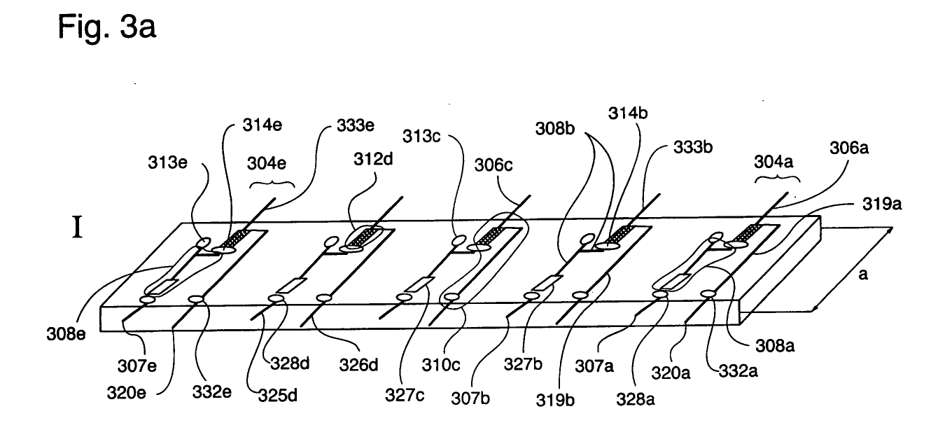

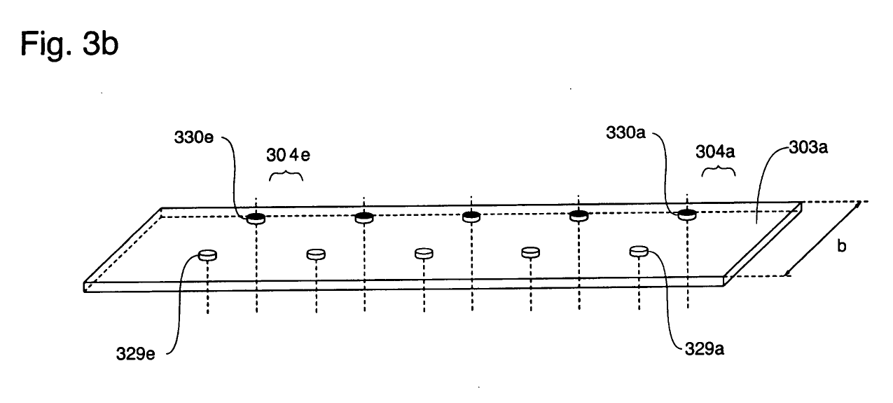

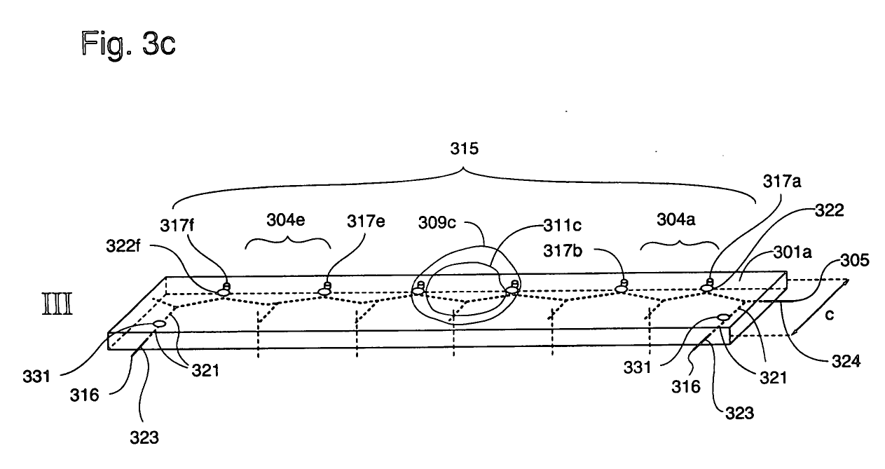

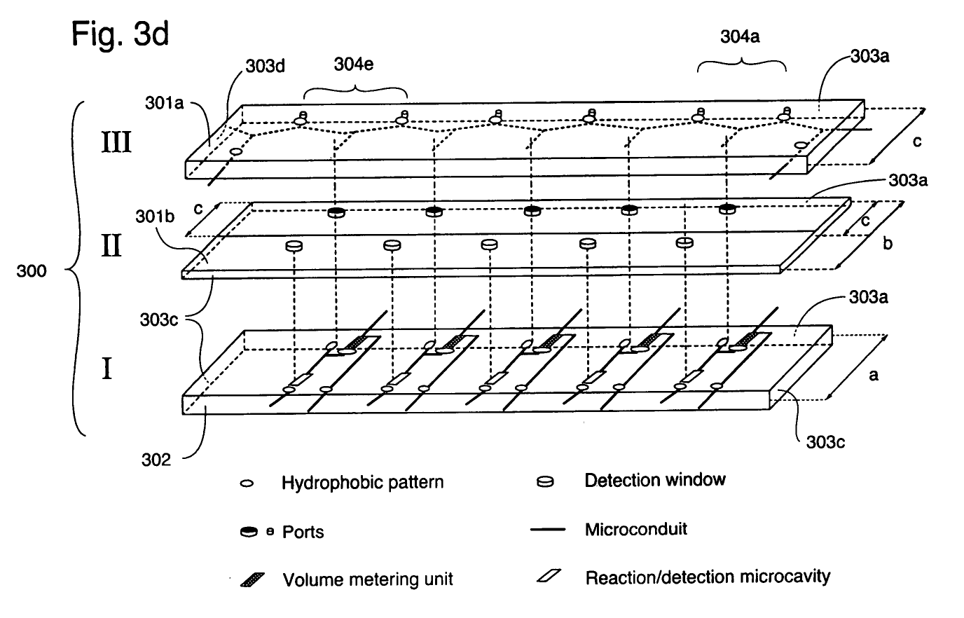

This aspect is illustrated in FIGS. 1 and 2. It comprises two main parts:
A) One or more essentially equal microfluidic devices (101a,b . . . ,201a,b . . . ), for instance of the kind (300) shown in FIG. 3. Each of the devices comprises a set (set I) of one or more essentially equal microchannel structures (304a,b . . . , FIG. 3) that are comprised within a common generally planar layer of the device (layer I). Each of the microchannel structures comprises an internal microconduit portion (308a,b . . . , FIG. 3) in which an active liquid flow is used for transport of liquid, reactants and the like in the downstream direction. Further details about layer I and the internal micro conduit portions (308) are given under the heading “Microfluidic device”.
B) An instrument (100,200), which comprises a spinner motor (102,202) and a rotary member (103,203). The instrument is intended for processing the microfluidic devices (101a,b . . . ,201a,b . . . ).

As illustrated in FIGS. 3a-c, the microchannel structures typically are composed of microstructures deriving from different substrate layers in case the device is constructed from two or more planar substrates that are joined together. For the variant shown in FIG. 3, layer I comprises at least the upper part of planar substrate I, whole planar substrate II, and the lower part of substrate III.

The rotary member (103,203) typically has an axis of symmetry (Cₙ with n≧2, 3, 4 etc up to ∞) that coincides with a spin axis (104,204). n are in preferred variants ≧5 including in particular circular variants (n=∞). The rotary member (103,203) is spun around the spin axis in a spin plane that is perpendicular to the spin axis.

The first aspect comprises two main characteristic features in combination.

The first main characteristic feature is that the rotary member (103,203) comprises a group (group A) of one or more seats (105a,b . . . ,205a,b . . . ) for retaining at least one of the microfluidic devices (101a,b . . . ,201a,b . . . ) on the rotary member. Each of the seats can i) be positioned at the same radial distance as any of the other seat of the group, and ii) align layer I essentially radially at an angle a relative to the spin plane where 0°<α≦90°, with preference for 45°≦α≦90°, such as α being essentially equal to 90° (as illustrated in FIGS. 1-2).

Essentially equal to 90° includes α that is in the interval 85°-90°.

For variants where layer I has an essentially radial orientation and α is essentially equal to 90°, an extension of the device inwards the spin axis (104,204) will typically intersect this axis or, when αis 90°, fully encompass it. In preferred variants this applies also to layer I. FIGS. 1-2 illustrate a variant where α is equal to 90°.

The arrangement may also comprise other kinds of microfluidic devices (not shown). In this case the rotary member may comprise separate groups of seats (group B, group C etc) for one or more of these other kinds of devices. Devices of different kinds may or may not fit into a specific group of seats.

In preferred variants, the microchannel structures of set I are essentially parallel. Each of the seats (105a,b . . . ,205a,b . . . ) of a group, in particular group A, can position corresponding parts of the internal microconduit portions (308a,b . . . , FIG. 3) of microchannel structures (304a,b . . . , FIG. 3) of set I in different microfluidic devices at essentially the same radial distance from the spin axis (104,204). This also applies for other sets (II, III etc) of microchannel structures, if present.

The second main characteristic feature is that the microconduit portion (308a,b . . . , FIG. 3) of each of the microchannel structures (304a,b . . . , FIG. 3) in each of the microfluidic devices (101a,b . . . ,201a,b . . . ) has an upstream part and a downstream part and an interconnecting part that permits liquid transport/flow from the upstream to the downstream part when the device (101a,b . . . ,201a,b . . . ) is placed in the rotary member (103,203) and spun around the spin axis (104,204). Typically, the upstream part is then at a shorter radial distance from the spin axis than the downstream part during.

The Instrument

The innovative instrument (100,200) illustrated in FIGS. 1-2 comprises a spinner motor (102,202) and a rotary member (103,203). The spinner motor comprises a shaft (106,206) with a spindle (107,207) through which the axis of symmetry/spin axis (104,204) passes, and is mounted on a frame (108,208). The rotary member (103,203) has on its upper side one or more seats (105a,b . . . ,205a,b . . . ) for holding a certain number of microfluidic devices (101a,b . . . ,201a,b . . . ) (ten and four in FIGS. 1 and 2, respectively). Each seat may hold one, two or more microfluidic devices.

Each microfluidic device shown in these figures has inlet ports in the form of protrusions (109,209). Each protrusion comprises an internal microconduit of capillary dimension and may be in the form of a tip separately attached to the surface of the microfluidic device. The protrusions in FIGS. 1-2 are attached to an edge side (=first edge side, 303a in FIG. 3d) of a microfluidic device that is disc-shaped.

The total number (x) of seats (105a,b . . . ,205a,b . . . ) that is possible on the rotary member (103,203) depends on the size of the rotary member, the size of the seats, the size of the microfluidic devices that are to be placed in the seats (thickness, extension in the radial direction etc), radial position of the seats, number of annular circles of seats/microfluidic devices, ability of the seats/devices to be rotated etc. Typical x may be found in the interval 2≦x≦1000, such as 2≦x≦100. This interval applies to many microfluidic devices that have a length to be oriented radially that is within the interval 2-30 cm.

Each seat (105,205) preferably is designed to secure that a microfluidic device placed in the seat can be retained while spinning the rotary member (103,203). This retaining function may, for instance, be a geometric configuration in the surface of the rotary member matching the part of a microfluidic device that is to be placed in the seat. Geometric configurations may be in the form of one or more grooves and/or one or more pins and/or other elevated and/or recessed structures. Sub-pressure and/or magnetic forces may also be used, typically in combination with geometric configurations and other retaining functions. There may be further functionalities for retaining the devices on the rotary member, for instance a top plate (110,210) that can be pressed to the upper parts of devices (101,201) that are placed in the seats (105,205) of the rotary member (103,203). This top plate (110,210) may comprise retaining functions on the side that is turned against the rotary member (typically the lower side of the top plate).

Retaining functions that are based on sub-pressure require introduction of sub-pressure on the rotary member (103,203). This is typically done from a non-rotary part of the innovative instrument, possibly involving also rotary parts other than the rotary member (103,203). Examples of other rotary parts are the spindle (106,206) and/or the shaft (107,207). The sub-pressure connection between a rotary part and a non-rotary part preferably a) provides low or no friction between these parts when spinning the rotary member and/or b) permits leakage of air between the rotary part and the non-rotary part concerned. A preferred variant is illustrated in WO 03024596 (Gyros AB). Also other kinds of connections may be used.

Retaining functions that are based on magnetic forces requires that either one or both of the microfluidic device (101a,b . . . ,201a,b . . . ) or the rotary member (103,203) with its seats (105,205) comprise magnetic or magnetizable material.

One or more of the seats may be designed to permit rotation of a microfluidic device (201) about an axis that is parallel to but remote from the spin axis (204) of the rotary member (203). This axis typically is unique for each seat and passes through the seat and/or a device placed in the seat. The rotation may be a full turn or a part of a turn. The rotation of the devices is in a plane that is parallel to the plane of the rotary member (spin plane). In a subvariant, one or more, preferably all, of the seats of a group may have two or more alternative retaining structures (e.g. geometric) that enables orientation of a microfluidic device at fixed angles relative to the radius passing from the centre of the rotary member through the seat concerned. Typically angles are 0°, 90° and/or 180°. A change in orientation of the device is typically accomplished manually. In another subvariant, each of the seats of a group is present on a separate turntable (211a,b . . . ) that is present on the rotary member (203) and can rotate independent of the spinning of the rotary member (203). The plane of rotation of a turntable is parallel to the spin plane of the rotary member. The turntables (211a,b . . . ) may be driven by an electric motor or manually. A change of orientation may be accomplished automatically according to a predetermined time schedule defined by the process protocol programmed into a controller of the instrument (not shown). The use of seats permitting separate rotation of microfluidic devices is in particular of importance for variants in which α is 90° or essentially equal to 90°.

The ability of rotating a device 180° as described in the preceding paragraph permits reversal of the flow direction. It thus becomes possible to transport a liquid (including dissolved reagents and dispensed particles) back and forth in a part of a microchannel structure.

One can also envisage that the possibility of reversing the liquid flow will make it possible with extended microchannel structures comprising extremely large number of functional units permitting more complex procedures without increasing the size of a device. A microchannel structure may thus start at one edge side, reach the opposite edge side where a reversing unit permits the microchannel structure to go back towards the starting edge side. Once the reagents/products etc that are under processing end up in the reversing unit the device is rotated 180° and the process continued.

In other variants the seats can be moved laterally, for instance in a radial direction. In these variants it may be possible to regulate the flow velocity in the internal microconduit portions (308) by moving the seats (105,205) in the radial direction. Presuming constant spin velocity, the flow velocity will increase when increasing the radial distance by moving a seat outwards (and the microfluidic device), and decrease when moving a seat inwards. By placing a number of essentially equal microfuidic devices at different radial distances a spectrum of flow velocities can be effectuated simultaneously.

The capability of radial movement of individual seats (105,205) will simplify individual process treatments of the devices (101,201). A device may thus from time to time be separately placed at a more outward position than the other devices on the rotary member (103,203). This will facilitate measurements, irradiations etc of part areas of individual microfluidic devices (101,201) that are present simultaneously on the rotary member (103,203).

The capability of radial movement also provide a simple way for the transfer of microfluidic devices between two properly aligned innovative instruments, for instance in order to reverse the flow without rotating a device 180°.

In still another subvariant, the seats may permit that microfluidic devices (101,201) placed in the seats (105,205) can be moved upwards and/or downwards in relation to the plane of the rotary member (103,203) (axial movement). The movement for a device/seat may be dependent or independent from the movement of the other devices/seats. This variant will also facilitate individual process treatments of the devices, e.g. measurement, irradiation etc.

The spinner motor (102,202) should be able to create the necessary centrifugal force for driving a liquid between an upstream position and a downstream position in the internal microconduit portions (308) of devices that are placed on the rotary member (103,203). Centrifugal force may be utilized in combination with a second liquid volume to create a sufficient local hydrostatic pressure within a structure to drive a first liquid volume through an outward (downward) and/or an inward (upward) bent of a microchannel structure. See for instance WO 0146465. The spinner motor (102,202) should be able to provide spin velocities that typically are within the interval 50-30000 rpm, such as 50-25000 rpm, or part(s) of these intervals. Spinner motors providing even higher spin velocities may be used. The spinner motor is preferably regulatable in the sense that the spin velocity can be set to different values and different accelerations and/or decelerations. Centrifugal force may also be combined with other forces and/or means to drive liquid flow in a microfluidic device.

The Microfluidic Device

The microfluidic device (300) is illustrated in FIG. 3 as a three-layered variant comprising three planar substrates (I, II, and III) where substrate I and substrate III have open microstructures in their upper and lower surfaces, respectively, and substrate II has holes passing through the substrate. When the substrates are apposed as suggested in FIG. 3d, the microchannel structures (304a,b . . . ) are formed. The widths (a, b and c) of substrates I, II and III of the variant of FIG. 3 are a=b>c. This means that the microfluidic device (300) typically comprises two essentially planar and parallel opposite sides (301a and 302 in FIG. 3d), and edge sides (303a,b . . . in FIG. 3d). The parallel opposite sides typically defines a top side (301a,b) and a bottom side (302) of the devices. The top side and/or the bottom side of the device are typically polygons, for instance with straight sides and perpendicular corners, such as in a rectangle, square etc (rectangular disc, square-shaped disc). Typically the top side and the bottom side have the same size and/or shape and are aligned in such a way that edge sides are perpendicular to the top and bottom side. The area of an edge side is typically smaller than the area of the top side and/or the bottom side.

The device (300) is preferably a disc or is disc-shaped. The disc may be planar in which is included variants in which planar substrates of different lengths and/or widths have been used in the manufacture as illustrated in FIG. 3d.

The number of microchannel structures (304a,b . . . ) per device (300) depends on the size of the device and/or the individual microchannel structures. Typically the microfluidic device comprises in total ≧2, such as ≧3 or ≧5 or ≧10 or ≧25 or ≧50 microchannel structures. A typical upper limit is between 100 and 1000, such as between 100 and 500 microchannel structures per device. The microchannel structures may be divided into sets (sets I, II, III etc) depending on design, direction in the device, layer in which they extend in the device etc. The number of microchannel structures in a set is typically within the interval of 1-50, such as 2-25 or 2-20. Microchannel structures of the same set may have a common inlet port, possibly associated with a common distribution manifold (see below). Many times there is only one set in each device. The microchannel structures of a set are typically essentially parallel. The layer (layer I) in which the microchannel structures of a set extend is typically parallel to the top side or to the bottom side of the device.

The prefix “micro” contemplates that each individual microchannel structure (304) comprises one or more microcavities and/or microconduits that have a depth and/or a width that is ≦10³ μm, such as ≦5×10² μm or ≦10² μm. Dimensions within this interval are preferably at hand in any location in a microchannel structure. The volume of a microcavity and thus also of liquid aliquots to be transported and processed are typically in the nl-range, i.e. <5,000 nl, such as ≦1,000 nl, or ≦500 nl or ≦100 nl or ≦50 nl or smaller. There may also be larger microcavities extending above the nl-interval, e.g. with volumes 1-10 μl, 1-100 μl, and 1-1,000 μl (μl-range). These larger microcavities are typically associated with inlet ports for liquid and used for the introduction of samples or washing liquids and the like.

Microcavities or microchambers may have the same or a different cross-sectional geometry compared to surrounding microconduits.

The microchannel structures are typically enclosed, e.g. covered, but have openings for inlet/outlet of liquid and/or air (ports/vents).

Different Parts of a Microchannel Structure

A microchannel structures (304) comprises the functional units that are necessary to carry out a predetermined process within the structure and therefore has at least:
a) one or more inlet arrangements each of which includes one or more inlet ports (e.g. IP I₁ and IP I₂; 305 and 306a,b . . . , respectively),
b) one or more outlet arrangement each of which includes one or more outlet ports (OP I₁, OP I₂, OP I₃; 307a,b . . . , 316, 320a,b . . . ), and
c) an internal microconduit portion (308a,b . . . ) between an inlet arrangement and an outlet arrangement.

An inlet arrangement typically comprises also a volume-metering unit for liquids (309a,b . . . and 310a,b . . . ; FIGS. 3c and 3a, respectively) from which a metered liquid volume is transported further downstream in the microchannel structure. As illustrated in FIG. 3 there may be two kinds of inlet arrangements:
1) a common inlet port IP I₁ (305) together with a common distribution system or distribution manifold (315) comprising several volume-metering units (309), and
2) a separate port IP I₂ (306a,b . . . ) and volume-metering unit (310a,b . . . ) for each microchannel structure.

An inlet arrangement may also comprise other functional units, e.g. a separation unit for removing particulate material upstream a volume-metering unit. Separation units for removing particulate material may be based on sedimentation, filtering etc.

An outlet arrangement may or may not be directly linked to a downstream end of an internal microconduit portion (308a,b . . . ). FIG. 3 illustrates three kinds of outlet arrangements:
1) an outlet port (OP I₁) (307a,b . . . ) that is in communication with the downstream end of the internal microconduit portion (308a,b . . . ) of a microchannel structure and used for the disposal of processed liquid aliquots,
2) an outlet port (OP I₂) (316) that is in communication with the distribution manifold (315) and used for the disposal excess liquid that has been dispensed to the distribution manifold (315), and excess air,
3) an outlet port (OP I₃) (320a,b . . . ) for the disposal of excess liquid that has been dispensed to a single volume-metering unit and is allowed to pass out via an overflow microconduit (319a,b . . . ) associated with the single volume-metering unit (310a,b . . . ).

An outlet arrangement may or may not comprise a waste treatment function. Outlet ports are typicallya also used as air vents or outlets of air

The internal microconduit portion (308a,b . . . ) typically comprises one or more functional units in which a liquid, such as a sample, is processed. In this portion an active liquid flow is typically used for transport of liquid, reactants and the like in at least a part of the portion.

An inlet port, such as IP I₁ (305) or IP I₂ (306), is primarily used as an inlet for liquid and/or particles (e.g. in suspensed form). An outlet port, such as OP I₁ (307), is primarily used as an outlet vent for air and liquid. Ports may also have other functions or combinations of functions, for instance selected from inlet for air (vent), outlet for air (vent), inlet for liquid, and outlet for liquid.

A functional unit (microconduit or system of microconduits) that is common to several microchannel structures is a part of each of the microchannel structures it is common to. Inlet port 305 and also the distribution manifold 315 in FIG. 3 are thus part of each microchannel structure they are communicating with.

A volume-metering unit is used to meter a part of a liquid volume that has been dispensed into the inlet port associated with the unit. The metered volume is then further transported downstream into the microchannel structure concerned from the inlet arrangement. The precision in the metering should be high, typically within the interval ±10%, such as within ±5%, around a predetermined volume.

A volume-metering unit (309a,b . . . ,310a,b . . . ) typically comprises a volume-defining microcavity (311a,b . . . (FIG. 3c) and 312a,b . . . (FIG. 3a), and a valve function (313a,b . . . (FIG. 3a) and 314a,b . . . (FIG. 3a)) that are associated with the lower end of the volume-defining microcavity (311a,b . . . and 312a,b . . . , respectively). The valve functions (313a,b . . . ,314a,b . . . ) prevent undesired leakage of liquid from the volume-metering units into downstream parts of the microchannel structure(s). Typically there may also be an overflow microconduit (321,319a,b . . . ) through which excess of air and/or of liquid is removed from the volume-metering units.

When two or more microchannel structures are associated with the same inlet port (305) for liquid, the volume-metering units (309a,b . . . ) may define a distribution manifold (315) that is common for the microchannel structures connected to the inlet port (IP I₁, 305). The distribution manifold (315) illustrated in FIG. 3 comprises a series of volume-metering units (309a,b . . . ) and one or more outlet ports (OP I₂, 316) for excess air and excess liquid (overflow microconduit). If necessary, inlet/outlet ports solely for air (inlet vents) (317a,b . . . ) may be located at critical positions in order to support an efficient partition of well-defined liquid volumes to each of the microchannel structures associated with the distribution unit. In the variant shown in FIG. 3, critical positions are between the volume-defining microcavities and/or on each terminal part (317b-e and 317a,f, respectively). The positions of these inlet vents are selected such that each inlet vent participates in defining the volume to be metered in the microcavity concerned, for instance such that the volume between each pair of close vents will define the volume to be metered. Anti-wicking structures, for instance in the form of local changes in the chemical or geometric surface characteristics may be present at the same positions as the inlet vents (317a,b . . . ) to assist in the volume-definition. In order to prevent undesired passage of liquid through these inlet vents, the inside of the venting microconduits of the inlet vents (317a,b . . . ) are typically hydrophobic (322a,b . . . ), in particular at their connection to parts that are to contain liquid.

When only one microchannel structure is associated with an inlet port (306), the volume-metering unit (310a,b . . . ) typically comprises a volume-defining microcavity (312a,b . . . ) which at its outlet end has a valve function (314a,b . . . ) and at its inlet end is connected to the inlet port (306a,b . . . ) via an inlet microconduit and to an overflow microconduit (319a,b . . . ) through which excess liquid can leave the main flow path. The cross-sectional area of the volume-defining microcavity (312a,b . . . ) is typically increasing at its inlet end where the over-flow microconduit (319a,b . . . ) is attached and decreasing at its outlet end. The overflow microconduit (319a,b . . . ) in the variant shown in FIG. 3 ends in an outlet port (OP I₃, 320a,b . . . ).

Microconduits (overlow microconduits) (321 and 319a,b . . . ) typically have valve functions (331 and 332a,b . . . , respectively).

In the preferred variants the volume-metering units are directed downwards with their connections (valves 313 and 314) to downstream portions of the microchannel structures (304a,b . . . ) at the lowest level of each unit. The outlet ends (316 and 320a,b) of the excess microconduits (321 and 319) are typically at a level that is lower than the inlet(s) vents (317a,b . . . ), e.g. lower than the connection between the corresponding volume-metering unit (309,310) and the corresponding downstream parts of the microchannel structure, i.e. between a volume-metering unit (309,310) and an internal microconduit portion (308).

The level at which the inlet ports for liquid is located is not critical as long as self-suction (capillarity) is relied upon for filling the volume-metering units.

A volume-metering unit is capable of metering a liquid volume within the interval/subinterval for volumes discussed elsewhere in this specification.

In order to prevent losses of metered liquids due to wicking, anti-wicking structures may be located between an inlet port for liquid and a volume-metering unit located downstream.

Further information on the design of distribution manifolds, volume-metering units, anti-wicking structures, valves, separation units for removing particulate material etc can be found in for instance WO 9853311 (Gamera Biosciences), WO 02074438 (Gyros AB), WO 0318198 (Gyros AB) and many others. See in particular units 3, 7, 10-12 (FIGS. 4, 8, 11-13) in WO 0274438 and units B-D (FIGS. 3-5) in WO 0318198 (Gyros AB) and other passages related to anti-wicking structures and valves in the publications cited in this specification.

Inlet ports that have the same function are typically present on the same side, for instance on an edge side (303) or on the top side or bottom side (301,302). Inlet ports having different functions are typically present on different sides, for instance on different edge sides (303a,b,c or d), or on an edge side and on one of the parallel opposite sides (301,302), or on the top side and the bottom side (301,302). An inlet port, such as OP I₁ (305) in FIG. 3, that is common for several micochannel structures may thus be on one side, and an inlet port, such as OP I₂ (306), that is only connected to a single microchannel structure or to another combination of microchannel structures on another one of the sides mentioned.

The opening of a port may be on a tip (323 and 324 in FIG. 3c; 325a,b . . . , 326a,b . . . , and 333 in FIG. 3a) that comprises a microconduit that ends in the opening. The tip may be in the form of a capillary tube, triangular etc and may be an integral part of or separately attached to the substrate(s) from which the microfluidic device is manufactured. A general term for this kind of port design is a protrusion. Alternatively a port may be an opening directly in the flat surface of the appropriate side of the device.

The protrusion design of the inlet ports is particularly well adapted to our innovative methodology herein called “Dip-Chip technique” which comprises that the loading of liquid is accomplished by simultaneously dipping ports of the same kind into a liquid to be introduced. If there are more than one inlet port of the same kind, the individual ports may be dipped simultaneously into separate liquids, for instance into wells of a microtitre plate. If the interior surface of the corresponding inlet arrangement(s) has/have a sufficient wettability as discussed elsewhere in this specification capillarity will cause the liquid to fill each of the microchannel structures (304) to the first valve function(s) (313 and 331 for inlet port 305, and 314 and 332 for inlet ports 306). Upon spinning the microfluidic device in the innovative instrument and opening the valve functions. (332 and 331) in the overflow micrconduits (319 and 332, respectively), excess liquid will leave the microchannel structures via the overflow microconduits (321 for inlet port 305, and 319 for inlet ports 306) leaving a well-defined volume of liquid in each of the volume-defining microcavities (311a,b . . . for inlet port 305, and 312a,b . . . for inlet ports 306). When increasing the spin velocity the liquid in the volume-defining microcavities will be transported further downstream to the reaction microcavities (327a,b . . . ). If each of the reaction microcavities ends with a valve function it will be possible to carry out a reaction at non-flow conditions. If the there is no valve function present the reaction is typically performed under flow conditions. Se further the publications cited as background publications, in particular WO 0275312 (Gyros AB) and WO 03093802 (SE 0201310-0) (Gyros AB).

Loading of ports that are plain openings in the flat surface of the device may be performed in 30 a conventional manner, typically by the use of pipettes and/or more or less automated dispensers. If the same liquid is to be introduced to all ports of a side, the side concerned may simply be dipped into the liquid.

In certain variants there may be a need for inlet portions of different microchannel structures to cross or intersect each other while keeping them physically apart in order to avoid unwanted mixing of liquids. This is the case for variants in which
A) each microchannel structure of a set is linked to two or more inlet ports for liquid and at least one of these ports is common to several microchannel structures of the set, and
B) at least two of the inlet ports that are connected to the same microchannel structure mouth in an edge side.

Placing crossing microconduit parts of different microchannel structures in different sublayers can avoid the risk of undesired mixing. This is illustrated in FIG. 3 where the upstream part of inlet arrangement(s) that comprises/comprise one kind of inlet port (for instance inlet port 305) is(are) in a sublayer that is physically separated from the sublayer comprising the upstream part of inlet arrangement(s) that comprise/comprises the other kind of inlet port(s) (for instance inlet port(s) 306). The upstream part in this context comprises at least an inlet port with its inlet microconduit and possibly also the corresponding volume-defining microcavity(ies) and/or any functional unit, such as a separation function, that is located between the inlet port and the volume-metering unit. The sublayer represented by the intermediate substrate II (FIGS. 3b and d) typically provides for liquid communication between microconduits that are present in different sublayers (e.g. substrates I and III) that are placed on different sides of an intermediary sublayer/substrate (e.g. substrate II).

The internal microconduit portion (308) may comprise one or more of the following functional units: microconduit for liquid transport, valve unit, branching unit, vent to ambient atmosphere (outlet port), unit for mixing liquids, unit for performing chemical reactions or bioreactions, unit for separating soluble or particulate material from a liquid phase, waste liquid unit including waste cavities and overflow channels, detection unit, unit for collecting an aliquot processed in the structure, possibly for further transfer to another device e.g. for analysis, branching unit for merging or dividing a liquid flow, etc. Units may be combined, for instance detecting/measuring may take place in a reaction microcavity, for instance via a transparent wall (detection window) of this microcavity. The presence of functional units in the internal microconduit portion is illustrated in FIG. 3 where each internal microconduit portion (308) has a reaction microcavity (327a,b . . . ). Depending on whether or not the reaction to be performed is to take place under non-flow conditions or flow conditions there may or may not be a valve function (328a,b . . . ) at the outlet end of the microcavity (327). By making the wall of the reaction microcavity translucent/transparent (detection window, 329a,b . . . ) it will be possible to measure results of events taking place within the reaction microcavity.

Further details about useful functional units can be found in the publications cited above, primarily with Gamera Biosciences/Tecan or Gyros AB/Amersham Biosciences as assignees.

A microchannel structures typically has a main direction of liquid flow (D1) which is defined as the direction from the start to the end of the internal microconduit portion (308a,b . . . ) regardless of turns, branches, parts where the liquid is taken back and forth etc. In the case there are no microchannel structures having other main directions of flow, D1 for a microchannel structure will also be the main direction of flow for the device concerned. In a typical case D1 is directed from one edge side (first edge side (303a)) to another edge side of the device, e.g. an opposite edge side (second edge side (303c)). In variants of the microfluidic device (300) that allow for reversal of liquid flow relative to D1, the main direction D1 is the main flow direction in the initial part of the internal liquid microconduit (308), typically up to the stage where reversal occurs.

If not otherwise is apparent from the context, terms such as “higher”, “upper” and “inner” level/position of a microchannel structure (304) are relative and means that the level/position concerned is located in a direction that is opposite to the main direction D1 compared to a level/position that is at a “lower” level/position. The terms “up”, “upward”, “inwards” etc and “down”, “downwards”, “outwards” will mean “against” and “along”, respectively, the main direction D1 of a microchannel structure.

The device (300) may be placed in a seat (105,205) on the rotary member (103,203). It is always possible to orient the disc plane outwards with the upstream part of the internal microconduit portion (308a,b . . . ) at a shorter radial distance than the downstream part. This orientation means that the first edge side (303a) becomes closest to the centre (axis of symmetry, spin axis) (104,204) of the rotary member (103,203). The main direction D1 of the device will be from the first edge side (303a) (the centre) to the opposite edge side (303c) (outwards), possibly at a certain angle (β) relative to the spin plane (−90°<β<90° with preference for −45°<β<45°, such as 0°).

Wettabillty/Non-Wettability of Inner Surfaces

The microchannel structures have in preferred variants inner surfaces that are hydrophilic. Hydrophilicity may be introduced, for instance as described in WO 0056808, WO 0147637, or U.S. Pat. No. 5,773,488 (Gyros AB). The hydrophilicity should be as given in these publications, i.e. the wettability of the interior of a structural unit should be sufficient for capillary forces to fill the unit with liquid once the liquid front has passed the inlet of the unit. Where appropriate hydrophobic surface breaks (e.g. as anti-wicking means and/or valves) are introduced as outlined in WO 9958245 and WO 0274438. See also WO 0185602 (Åmic AB & Gyros AB).

The exact demand on hydrophilicity (liquid contact angles) of inner surfaces of the microchannel structure may vary between different functional units. Except for local hydrophobic surface breaks the liquid contact angel for at least two or three inner walls of a microconduit in a particular unit should be wettable (=hydrophilic=liquid contact angle≦90°) for the liquid to be transported, with preference for liquid contact angels that are ≦60°, such as ≦50° or ≦40° or ≦30° or ≦20°. In the case one or more walls have a higher liquid contact angle, for instance is non-wettable (hydrophobic), this can be compensated by a lowered liquid contact angle on the remaining walls. This may be particularly important if non-wettable lids are used to cover open hydrophilic microchannel structures. The values above apply to the temperature of use. The liquid referred to is typically water including also other aqueous liquids.

The liquid contact angles given above refer to equilibrium contact angles and measured at the temperature of use, for instance room temperature such as +25° C.±5° C.

What has been said above about hydrophilicity/hydrophobicity applies in particular to the inlet arrangement of the microchannel structures (304) in the preferred microfluidic devices (300), including also the tip part of the microchannel structures, if present.

Microconduits that are used solely for venting purposes (inlet and/or outlet venting) typically have hydrophobic inner surfaces at least at their connection to a microconduit intended for liquid.

Valve Functions

Valve functions can typically be selected from three main categories:
1. Mechanical valves.
2. Valves that comprise intersecting channels together with means that determine through which channel a liquid flow shall be created.
3. Inner valves, i.e. valves in which the passage or non-passage of a liquid depends on physical and/or chemical properties of the liquid and the material in the surface of the inner wall at the valve.

Type 1 valves typically require physically closing of a microconduit and are therefore called “closing valves”. They often have movable mechanical parts.

Type 2 valves function without closing and are therefore “non-closing”. A typical example is directing an electrokinetic flow at the intersection of two channels by switching the electrodes. See for instance U.S. Pat. No. 5,716,825 (Hewlett Packard) and U.S. Pat. No. 5,705,813 (Hewlett Packard).

In type 3 valves, the non-passage or passage of a liquid may be based on:
(a) changing the cross-sectional area in a microconduit at the position of the valve function by changing the energy input to the material of the wall in the microconduit (closing valves), and/or
(b) providing a boundary between surfaces of different interaction energy with a through-flowing liquid at the valve position (non-closing, capillary or passive valves), and/or
(c) a suitable curvature of the microconduit at the valve function (geometric valves, non-closing).

Type 3a valves are illustrated in WO 0102737 (Gyros AB) in which stimulus-responsive polymers (intelligent polymers) are suggested to create a valve function, and in WO 9721090 (Gamera Biosciences) in which relaxation of non-equilibrium polymeric structures and meltable wax plugs are suggested as valves.

Type 3b valves typically are based on local changes in chemical and/or geometric surface characteristics. Through-flow is achieved by increasing the force driving the liquid. The use of hydrophobic surface breaks (changes in chemical surface characteristics) as valves has been described in WO 9958245, (Gyros AB) WO 0146465 (Gyros AB), WO 0185602 (Åmic AB & Gyros AB), WO 0187486 (Gyros AB) and WO 0274438 (Gyros AB) and WO 031898 (Gyros AB). The use of changes in geometric surface characteristics as valves has been described in WO 9615576 (David Sarnoff Res. Inst.), EP 305210 (Biotrack), and WO 9807019 (Gamera Biosciences). Other alternatives are a porous membrane having pores or clusters of small holes that require a sufficient driving force for the liquid to pass through. The pores/holes are typically hydrophobic and have sizes corresponding to circular areas with a diameter ≦5 μm such as ≦1 μm.

Type 3b valves often comprise an anti-wicking function if they utilize changes in chemical and/or geometrical surface characteristics in edges as described for anti-wicking structures.

Type 3c valves for centrifugal based systems may be achieved by linking the downstream end of a downwardly bent microconduit (U- or Y-shaped) to an upwardly bent microconduit. This is illustrated in WO 0146465 (Gyros AB) with two or more Y/U-shaped structures in sequence in the downstream direction.

If a closing valve is used in a microfluidic device, there is typically an outlet vent associated with the upstream end of the valve function.

Anti-Wicking Structures

Anti-wicking structures are typically local surface modifications that counteract wicking, i.e. undesired liquid transport in the inner edges of microconduits. In microfluidic devices anti-wicking structures are particularly important when retaining liquid volumes that are in the nl-range within predetermined microcavities.

An anti-wicking structure typically comprises a change in surface characteristics in an inner length-going edge of a microconduit. The edge typically starts in a microcavity and stretches into a microconduit connected to the microcavity. The change may relate to a change in geometric and/or chemical surface characteristics. Anti-wicking structures may be present upstream or downstream a microcavity intended to contain a liquid. An anti-wicking functionality may inherently also be present in inner valves that are based on the presence of a hydrophobic surface break in an inner edge.

A change in geometric surface characteristics is typically local and may be selected from deformations, such as indentations and protrusions (projections). In most cases the deformation will also stretch into and across an inner wall of the microconduit concerned. See further WO 0274438 (Gyros AB) and WO 031898 (Gyros AB).

Deformations in the form of indentations, for instance in the form of “ear-like” or triangular, trapezoidal etc grooves as illustrated in FIGS. 3, 5, 8, 10, 11, 12 and 13 of WO 0274438, in FIGS. 2, 4 and 5 of WO 031898 (Gyros AB), and in FIG. 1 of WO 0275312 (Gyros AB).

A change in chemical surface characteristics (surface break) for anti-wicking purposes means in a typical case that the inner surface of a wettable microconduit comprises regions that are non-wettable. These regions are primarily present in inner edges of the microconduit but will in the preferred cases extend fully between edges.

A change in geometric and a change in chemical surface characteristics may fully or partially coincide in the inner surface of microconduit.

Further information about various kinds of anti-wicking structures possibly combined with an inner valve function is given in WO 0274438 (Gyros AB) and in WO 031898 (Gyros AB).

Manufacture of the Microfluidic Device.

The microfluidic device may be manufactured from inorganic or organic material. Typical inorganic materials are silicon, quartz, glass etc. Typical organic materials are plastics including elastomers, such as rubber silicone polymers (for instance poly dimethyl siloxane) etc. In a preferred variant, open microstructures are formed in the surface of a planar substrate by various techniques such as etching, laser ablation, lithography, replication etc. From the manufacturing point of view, plastic material are preferred and the microstructures, typically in the form of open microchannels are formed by replication, such as embossing, moulding, casting etc. The microstructures are then covered by a top substrate that if required also is microstructured. See for instance WO 9116966 (Pharmacia Biotech AB). The microstructures in the substrates are designed such that when the surfaces of two planar substrates are apposed the desired enclosed microchannel structure is formed between the two substrates.

Microfluidic devices that require that different parts of a microchannel structure are in different sublayers of layer I (=the layer in which the microchannel structures of set I extend) may be formed by including several substrate layers in the manufacturing method. See FIG. 3 and the text above. A common inlet arrangement (305+309) as an uncovered microstructure ay thus be defined in the surface of a first substrate (III, as indicated in the bottom side of the substrate) and the parts of the microchannel structures (304) that are not defined in the first substrate (III) may be defined in the surface of one or more additional planar substrates (I, as indicated in the top side of the substrate). The microstructures in the substrates match each other such that when the substrates are apposed and joined together the microfluidic device with its microchannel structures will be formed. If needed there may be an intermediary planar substrate (II) placed between two juxta-positioned substrates. An intermediary planar substrate (II) that provides for liquid communication (330a,b . . . ) between parts of the microchannel structures that are defined in different substrates (III and I). A hole, or a cluster of smaller holes or a porous membrane are typically present at the locations where liquid communication is to take place. FIG. 3 also illustrates that a planar disc may be manufactured from planar substrates having different forms (in this case breadths). In FIG. 3 the widths (a and b) of substrate III and II are equal and larger than the width (c) of substrate I.

At the priority date of this invention the preferred plastic material was a) polycarbonates and plastic material comprising polyolefines. Polyolefins in this context are polymers comprising repeating hydrocarbon monomeric units which preferably consist of one or more polymerisable carbon-carbon doubles or triple bonds and saturated branched straight or cyclic alkyl and/or alkylene groups. Typical examples are Zeonex™ and Zeonor™ from Nippon Zeon, Japan, with preference for the latter. See for instance WO 0056808 (Gyros AB).

The Second Main Aspect—The Instrument.

The instrument may be used in the innovative arrangement. The main characteristic feature is that the instrument comprises the kind of seats (105a,b . . . ,205a,b . . . ) discussed for the first aspect of the invention. In other words the rotary member comprises seats, each of which is capable of orienting layer I of a microfluidic device (300) in the same manner as for the first aspect of the invention. Different variants are apparent from the description above and concern both the instrument as such and features of the instrument that are related to the microfluidic device to be used.

The Third Main Aspect—The Method/Use of the Instrument for Processing Two or more Microfluidic Devices in Parallel.

This method/use comprises the steps of:
i) providing an instrument (100,200) of the second aspect of the invention,
ii) providing one or more microfluidic devices (101,102,300) that are adapted to be retained in the seats (105,205) of the instrument (300),
iii) loading the necessary liquids, reactants, samples etc into one or more of the microchannel structures (304) of each of the microfluidic devices (300) provided in step (ii),
iv) placing the devices that has been loaded in step (iii) in the seats (105,205) of the rotary member (103,203), and
v) processing the devices that have been placed in the instrument in step (iv) by the use of at least one substep in which a liquid flow is created in parallel in the microchannel structures of the devices by spinning the rotary member (103,203) around its spin axis (104,204).

Step iii) may be carried out before and/or after step iv). Reactants and other necessary chemicals may also be predispensed to the microchannel structures (304), i.e. be included in the devices provided in step ii). If the microfluidic devices allow for it they may be loaded as outlined by the innovative “Dip-Chip” technique. See the fifth aspect of the invention. The innovative method may be part of a protocol comprising several additional process steps within or external to the instrument. Such external process steps may take place prior to or after steps i)-v) or as a step inserted into this sequence of steps.

The Fourth Main Aspect—a Microfluidic Device.

This aspect is a variant of the microfluidic devices generally described above as a part of the innovative arrangement. The main characteristic feature of the fourth aspect is a) that there is one, two or more inlet ports (305,306) in an edge side of the device (300), and b) that the hydrophilicity in the most upstream part of each of the microchannel structure(s) (304a,b . . . ) that is connected to this/these inlet port/ports is/are such that at least a predetermined volume of liquid is capable of penetrating this part in each microchannel structure by self-suction (capillarity). Each inlet port (305,306) may be in the form of a protrusion (324,333a,b . . . ) comprising a microconduit as described elsewhere herein. This volume may differ between the inlet ports of a set, for instance set I. It typically is at least the sum of the volume-defining cavities (311,312) in the volume-metering unit/units (309,310) which is/are associated with the inlet port concerned (305,306). See under the heading “Microfluidic devices” above.

In preferred variants the characteristic feature is that the corresponding parts of the internal microconduit portion (308a,b . . . ) of each of the microchannel structures are at essentially the same distance from said first edge side (303a) or at the same level.

Further characteristic features of the innovative microfluidic device has been described above in the context of the first aspect of the invention.

The Fifth Main Aspect—Loading by Dip-Chip Technique.

This aspect relates to a method for loading a microfluidic disc with liquid. The characteristic feature comprises the steps of:
(i) providing the microfluidic device of the fourth aspect of the invention;
(ii) providing the liquids to be introduced through each kind of inlet port(s),
(iii)dipping at least one kind of inlet port into the liquid under sufficient time for the predetermined volume for this kind of port to be sucked into the microchannel structures, and
(iv) defining a volume of the introduced liquid in each volume-metering associated with the inlet port(s) used for the introduction of the predetermined volume.

Step (iv) may be performed by utilizing centrifugal force for driving the liquid flow, for instance in an instrument of the present invention. Other driving forces may also be utilized by appropriately adapting the microfluidic device to the instrument and driving force utilized.

In the case the kind of port utilizes comprises two or more ports and different liquids are to be introduced through each of the ports, these liquid are preferably provided in separate vessels, for instance in wells of a microtitre plate. In this case the distances between the ports and/or between the wells are adapted to fit each other. Other ports may be used in the similar manner if they are adapted to the Dip-Chip technique. Alternatively there may be ports that are adapted to conventional dispensation techniques, such as by drop dispensers, pipettes etc.

Subsequent to step (iv) the metered volumes are transported further downstream in parallel in the microchannel structures associated with the kind of inlet port(s) used.

### Best Mode Embodiment

At the priority date the best mode embodiment corresponds to the variant shown in the drawings.

The invention is further defined in the appending claims that are part of the specification.

1. Microfluidic arrangement which comprises
A) one or more microfluidic devices, each of which comprises a set (set I) of one or more essentially equal microchannel structures that are comprised within a common generally planar layer of the device (layer I),
each of said microchannel structures comprises an internal microconduit portion in which an active liquid flow is used; and
B) an instrument, which is intended for processing said one or more microfluidic devices and comprises a spinner motor and a rotary member;
characterized in that
I) said rotary member comprises a group of one or more seats for holding at least one of said one or more microfluidic devices, each of said seats is capable of
i) being positioned at the same radial distance as any of the other seats of the group,
ii) aligning layer I essentially radially at an angle ax relative to the spin plane where 0°<α≦90°, with preference for cc being essentially equal to 90°, and
iii) preferably positioning the corresponding positions in said microconduit portion of said microchannel structures in any of said one or more microfluidic devices at essentially the same radial distance,
II) said internal microconduit portion has an upstream part that can be positioned at a shorter radial distance than a downstream part when the corresponding microfluidic device is placed in any of said one or more seats.

2. The arrangement of claim 1, characterized in that the seats are adjustable in the radial and/or the axial direction.

3. The arrangement of claim 1, characterized in that the seats are at a fixed radial position.

4. The arrangement of any of claims 1-3, characterized in that each of said devices has two planar surfaces that are parallel to layer I and typically are rectangular with preference for each of said devices being disc-shaped.

5. The arrangement of claim 1, characterized in that the seats are capable of holding layer I of each of the microfluidic devices at different angles relative to the radius passing through the seat concerned, for instance at angles of 0°, 90° and/or 180°.

6. The arrangement of any of claims 1-5, characterized in that the microfluidic device is according to any of claims 7-19.

7. A microfluidic device comprising
i) two essentially planar and parallel opposite sides, and edge sides,
ii) a set of one, two, three or more essentially equal microchannel structures, each of which comprises a first inlet arrangement comprising an inlet port IP I₁,
characterized in that
a) each of the inlet ports is present in an edge side, and
b) the wettability of the inner walls of said first inlet arrangement permits penetration by self-suction (capillarity) of at least a predetermined first volume of an aqueous liquid which is contacted with said one or more inlet ports.

8. The microfluidic device of claim 7, characterized in that said first inlet arrangement is common for more than one of the microchannel structures, such as all microchannel structures of the set.

9. The microfluidic device of claim 7, characterized in that
a) each of said microchannel structures comprises a second inlet arrangement comprising an additional inlet port IP I₂ which inlet arrangement and inlet port are connected to only one of the microchannel structures or is common for two or more microchannel structures,
b) the wettability of the inner walls of the second inlet arrangement permits penetration by self-suction (capillarity) of at least a predetermined second volume of an aqueous liquid which is contacted with IP I₂.

10. The microfluidic device of claim 7, characterized in that either one or both of IP I₁ and IP I₂, if present, is/are part of a protrusion that is integral with or extends from the surface of the device.

11. The microfluidic device of claim 7, characterized in that
a) at least one of said first and/or said second inlet arrangement, if present, comprises one volume-metering unit per microchannel structure associated with the arrangement, and
b) said volume-metering unit has an outlet end associated with a valve function, preferably passive, which controls liquid transport through said outlet end into downstream parts of the microchannel structure that is associated with the volume-metering unit.

12. The microfluidic device of claim 11, characterized in that
a) the inlet port of either one or both of said first and second inlet arrangements, if present, is fluidly connected to only one microchannel structure and
b) the volume-metering unit preferably has an overflow channel for defining the volume to be metered in the unit.

13. The microfluidic device of claim 11, characterized in that the inlet port of either one or both of said first and second inlet arrangements if present, is fluidly connected to two or more of the microchannel structures via a distribution manifold containing one volume-metering unit per microchannel structure that is in fluid communication with the inlet port.

14. The microfluidic device of claim 13, characterized in that said distribution manifold comprises an excess microconduit that is common for all the volume-metering units of the manifold.

15. The microfluidic device of claim 9, characterized in that said wettability/hydrophilicity is present from IP I₁ or IP I₂, if present, to said valve function in each volume-metering unit connected to the inlet port concerned, thereby permitting filling by capillarity said inlet part to said valve function with said aqueous liquid.

16. The microfluidic device of claim 11, characterized in that
a) each of the volume-metering units is capable of metering a liquid volume in the nanolitre range, e.g. ≦5000 nl such as ≦1000 nl or ≦500 nl or ≦100 nl, and
b) each of said predetermined first and second (if present) volume is essentially equal to the sum of the volumes of liquids to be metered in the volume-metering units associated with the inlet arrangement/inlet port concerned.

17. The microfluidic device of claim 7, characterized in that the inlet port(s) (IP I₁) of the first inlet arrangement(s) is(are) present on one side, and the inlet port(s) (IP I₂) of the second inlet arrangemnt(s), if present, is(are) present on a different side, preferably at least one of the IP I₁s and IP I₂s is present on an edge side or on different edge sides.

18. The microfluidic device of claim 7, characterized in that it is manufactured from at least two essentially planar substrates, one, two or more of which define the individual microchannel structures.

19. The microfluidic device of claim 7, characterized in that
(i) each of said microchannel structures extends in a layer of the device which layer is essentially parallel with said two opposite sides,
(ii) each of said microchannel structures comprises downstream one to said inlet arrangements an internal microconduit portion in which active fluid flow can be used for the transportation of liquid, reagents, analytes and the like, and preferably corresponding parts of the microconduit portion of each of said microchannel structures are at essentially the same distance from said first edge side.
